(19) United States
(12) Patent Application Publication
(10) Pub. No.: US 2023/0310047 A1
(43) Pub. Date: Oct. 5, 2023

(54) BONE WEDGE IMPLANT AND METHOD

(71) Applicant: Medartis AG, Switzerland (CH)

(72) Inventors: Matt COYNE, Warsaw, IN (US); Adam FINLEY, Warsaw, IN (US); Ryan SCHLOTTERBACK, Warsaw, IN (US); Brad BOYSEN, Warsaw, IN (US)

(73) Assignee: Medartis AG, Switzerland (CH)

(21) Appl. No.: 18/158,548

(22) Filed: Jan. 24, 2023

Related U.S. Application Data
(60) Provisional application No. 63/362,221, filed on Mar. 31, 2022.

(51) Int. Cl. A61B 17/80

(52) U.S. Cl. CPC A61B 17/8095; A61B 17/8061

(57) ABSTRACT

An osteotomy implant device including a tapered wedge, a first cylindrical post spaced from a distal side of the tapered wedge, a second cylindrical post spaced from the proximal side of the tapered wedge, and a middle bridge spanning lengthwise from the first cylindrical post to said second cylindrical post.

### CROSS REFERENCE TO RELATED APPLICATIONS

The present application claims priority to U.S. Provisional Application Ser. No. 63/362,221 filed on Mar. 31, 2022 (Attorney Docket No. 3768.126P1), which is incorporated herein by reference in its entirety.

### BACKGROUND

### Field of the Invention

This application relates to devices, and methods associated with surgical instruments, and more particularly devices, and methods for bone implants.

### Description of the Related Art

Foot anatomy and the proper positioning of bones in the foot are important for providing proper weight bearing, balance, and mobility. Pes planus, commonly known as flatfoot, is a postural deformity that occurs when the arch of the foot is absent while standing and can be painful when performing physical activity such as standing for long periods of time, walking long distances, and/or running. A variety of foot problems, e.g. improper bone development, posterior tibial tendon dysfunction (PTTD), arthritis, injury, or diabetic collapse may cause flatfoot. Often, orthotics, braces, and/or physical therapy are used to treat flatfoot. However, in some cases, additional surgical procedures may be needed to correct anatomical abnormalities associated with flatfoot and provide proper bone positioning to reduce pain and improve mobility.

Osteotomies, surgical procedures in which a bone is cut to shorten, lengthen, or otherwise change bone alignment, are often used to treat flatfoot. Common surgical techniques to treat flatfoot include the Evans calcaneal osteotomy or the Cotton osteotomy, which are osteotomies used to open bones and position autogenous bone (bones harvested from the patient's body), allografts (bones obtained from a bone bank), or synthetic wedges into the bone openings. The Evans calcaneal osteotomy may be used to insert a trapezoidal-shaped implant, i.e. wedge, into the lateral side of the calcaneus, i.e. heel bone, to provide lateral column lengthening. The Cotton osteotomy, which is a medial opening wedge osteotomy, may be used to insert a trapezoidal-shaped implant, i.e. wedge, into the medial cuneiform, which is a bone situated at the medial side of the midfoot, to reduce midfoot abduction.

Often the bones apply high compressive forces to the surgically inserted wedges. In order to maintain a desired wedge position, existing processes use various hardware, such as surgical staples, metal screws and plates, to lock the wedge into place. However, in some patients, the hardware used to maintain the wedge position, such as staples, screws, and plates, may become prominent or irritate nearby tendon or other soft tissue, which may lead to swelling and inflammation and instability of the bones.

Another problem with current devices is the inability for the device to be removed post-operatively. For example, once the device is implanted and bony fusion has occurred across the osteotomy, the current devices used are not designed to be able to be removed without cutting the device out of the bone and removing the bony fusion.

Thus, a need exists for improved devices, and methods for treating foot deformities such as flatfoot.

### SUMMARY OF THE INVENTION

Briefly, an osteotomy implant device constructed in accordance with one or more aspects of the present invention provides, for example, an improved device for treating foot deformities such as, for example, flat foot.

In one embodiment, an osteotomy implant device constructed in accordance with one or more aspects of the present invention comprises a tapered wedge, a first cylindrical post, a second cylindrical post and a middle bridge. The tapered wedge includes a medial portion and a lateral portion. The lateral portion includes an outer lateral side, a first appendage and a second appendage. The first appendage extends from a first end of the medial portion to the lateral side. The second appendage extends from a second end of medial portion to the lateral side. The first appendage, the medial portion and the second appendage form a U-shaped body. The U-shaped body defines an outer perimeter side, an inner perimeter side, a distal side extending between the outer perimeter side and the inner perimeter side, and a proximal side opposite the distal side and extending between the outer perimeter side and the inner perimeter side. The inner perimeter side defines a central opening. The distal side extends along the first and second appendages and the medial portion and the proximal outer side extend along the first and second appendages and the medial portion. The first cylindrical post is spaced from the distal side of the U-shaped body of the tapered wedge. The second cylindrical post is spaced from the proximal side of the U-shaped body of the tapered wedge. The middle bridge spans lengthwise from the first cylindrical post to the second cylindrical post. A first portion of the middle bridge extends from the distal side of the tapered wedge to the first cylindrical post, and a second portion of the middle bridge extends from the proximal side of the tapered wedge to the second cylindrical post.

In another embodiment, the osteotomy implant device constructed in accordance with one or more aspects of the present invention may be part of an osteotomy implant system that also includes an installer instrument removeably affixed to the medial portion of the tapered wedge to facilitate insertion of said tapered wedge into an osteotomy and a trial and bone preparation instrument.

In one example, the trial and bone preparation instrument may include a guide body and a trial portion. The trial portion includes a tapered wedge portion that mimics the tapered wedge for insertion into the osteotomy. The guide body includes a first bore, a second bore and a milling slot extending between the first bore and the second bore. The first bore, the second bore and the milling slot are configured to prepare the osteotomy for the osteotomy implant device.

These, and other objects, features and advantages of this invention will become apparent from the following detailed description of the various aspects of the invention taken in conjunction with the accompanying drawings.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only. In the drawings:

FIG. 1 is a first perspective view of one embodiment of a bone wedge implant constructed in accordance with an aspect of the present disclosure;

FIG. 2 is a front or proximal view of the bone wedge implant illustrated in FIG. 1;

FIG. 3 is a left side view of the bone wedge implant illustrated in FIG. 1;

FIG. 4 is a rear or distal view of the bone wedge implant illustrated in FIG. 1;

FIG. 5 is a top side view of the bone wedge implant illustrated in FIG. 1;

FIG. 6 is right side view of the bone wedge implant illustrated in FIG. 1;

FIG. 7 is a bottom view of the bone wedge implant illustrated in FIG. 1;

FIG. 8 is a first perspective view of one embodiment of a bone wedge implant constructed in accordance with an aspect of the present disclosure;

FIG. 9 is a front or proximal view of the bone wedge implant illustrated in FIG. 8;

FIG. 10 is a left side view of the bone wedge implant illustrated in FIG. 8;

FIG. 11 is a rear or distal view of the bone wedge implant illustrated in FIG. 8;

FIG. 12 is a top side view of the bone wedge implant illustrated in FIG. 8;

FIG. 13 is right side view of the bone wedge implant illustrated in FIG. 8;

FIG. 14 is a bottom view of the bone wedge implant illustrated in FIG. 8;

FIG. 15 is a perspective view of one embodiment of an installer constructed in accordance with one or more aspects of the present disclosure;

FIG. 16 is another perspective view of the installer shown in FIG. 15;

FIG. 17 is a right side view of the installer shown in FIG. 15;

FIG. 18 is an end view of the installer shown in FIG. 15;

FIG. 19 is a top view of the installer shown in FIG. 15;

FIG. 20 is a left side view of the installer shown in FIG. 15;

FIG. 21 depicts a perspective view of a patient's foot with the bone wedge implant embodiment illustrated in FIG. 1 implanted into the lateral side of the calcaneus, in accordance with one or more aspects of the present disclosure;

FIG. 22 depicts a perspective view of a patient's foot with the bone wedge implant embodiment illustrated in FIG. 1 implanted into the medial cuneiform, in accordance with one or more aspects of the present disclosure;

FIG. 23 is a perspective view of one embodiment of an instrument to facilitate bone preparation for a bone wedge implant constructed in accordance with one or more aspects of the present disclosure;

FIG. 24, is another perspective view of the instrument illustrated in FIG. 23;

FIG. 25 is a rear view of the instrument illustrated in FIG. 23;

FIG. 26 is a side view of the instrument illustrated in FIG. 23;

FIG. 27 is a front view of the instrument illustrated in FIG. 23;

FIG. 28 is a top view of the instrument illustrated in FIG. 23;

FIG. 29 is a perspective view of a patient's foot with the instrument embodiment illustrated in FIGS. 23-28 positioned into the lateral side of the calcaneus, in accordance with one or more aspects of the present disclosure; and

FIG. 30 depicts one embodiment of a surgical method of use of a bone wedge implant constructed in accordance with one or more aspects of the present invention.

### DETAILED DESCRIPTION OF THE EMBODIMENTS

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention.

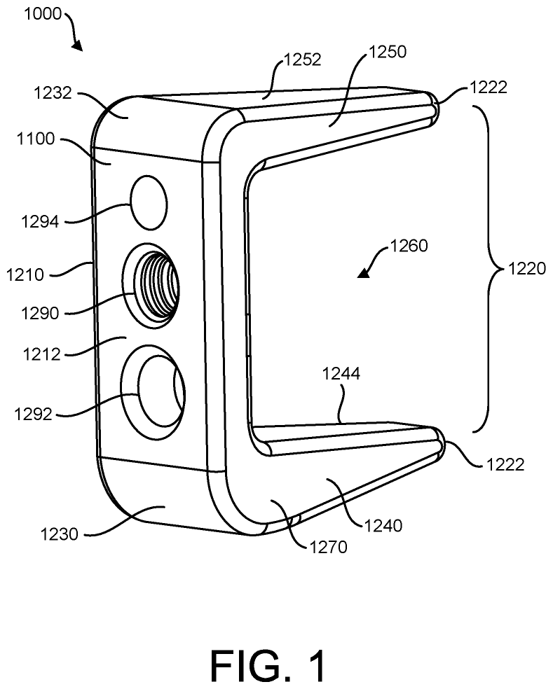

Thus, all the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in FIG. 1.

The following description references systems, methods, and apparatuses for cutting tools for orthopedic surgery involving a foot or lower extremities. However, those possessing an ordinary level of skill in the relevant art will appreciate that other extremities, joints, and parts of the musculoskeletal system are suitable for use with the foregoing systems, methods and apparatuses. Likewise, the various figures, steps, procedures and workflows are presented only as an example and in no way limit the systems, methods or apparatuses described to performing their respective tasks or outcomes in different timeframes or orders. The teachings of the present invention may be applied to any orthopedic surgery, such as on the hand as well as other upper and lower extremities and may be implemented in other treatments sites that have similar anatomical considerations.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

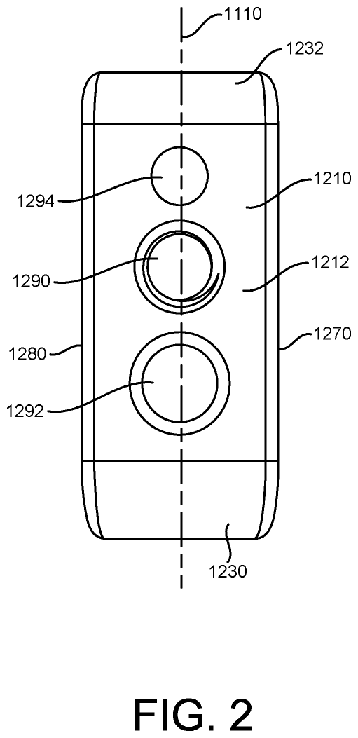

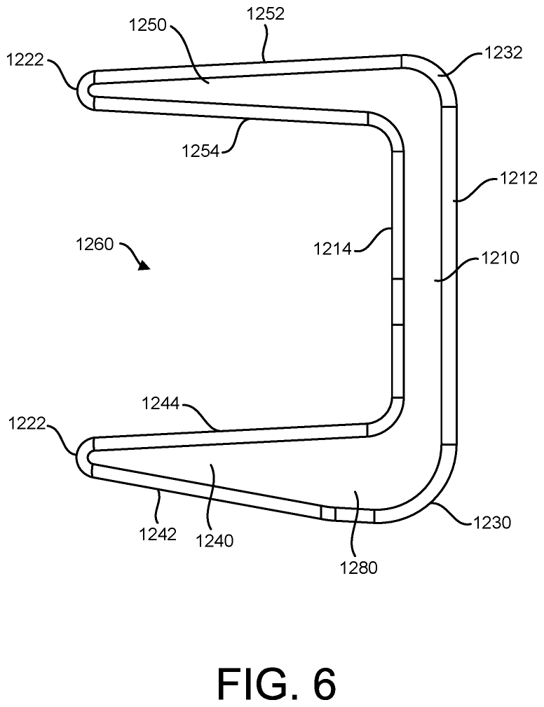

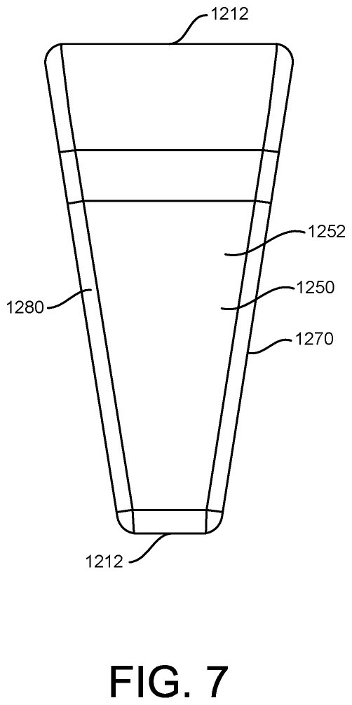

As will be described below, the present invention includes devices and methods for correcting a deformity of the human foot. As depicted in FIGS. 1-7, one embodiment of a bone wedge implant 1000 is presented. Bone wedge implant 1000 may be generally described as a horseshoe or U-shaped configuration designed to generally conform to an anatomical cross section of, for example, the medial cuneiform, or the lateral side of the calcaneus to provide mechanical support or other bone or joint in which it will be implanted.

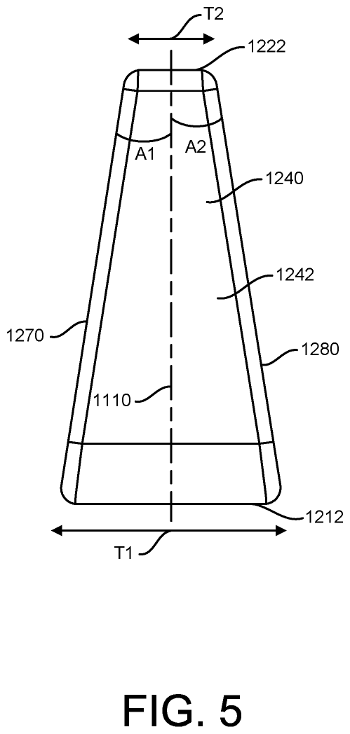

In one example illustrated in FIGS. 1-7, bone wedge implant 1000 includes a wedge 1110 having a body 1200. A top view of bone wedge implant 1000, shown in FIG. 5, shows that body 1200 of wedge 1100 is generally U-shaped or horseshoe shaped with rounded corners 1230, 1232.

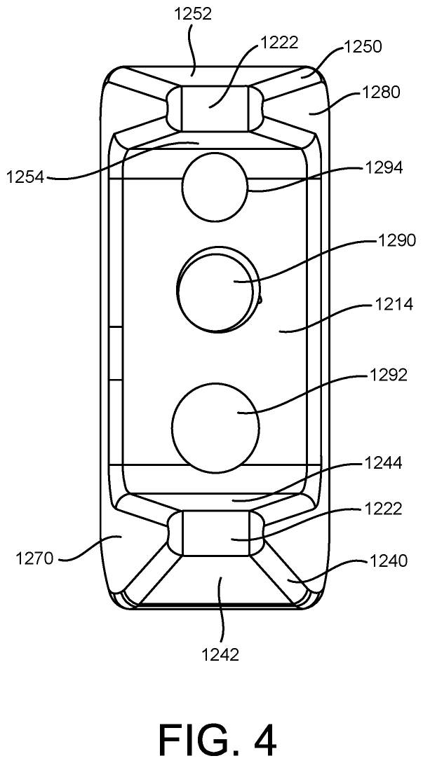

As illustrated in FIGS. 1 and 4, body 1200 of wedge 1110 is tapered from a medial portion 1210 to a lateral portion 1220. In other words, the thickness of body 1200 continuously decreases from medial portion 1210 to lateral portion 1220. For purposes of illustration, FIG. 5 shows body 1200 of wedge 1100 having a first thickness T1 adjacent medial side surface 1212, which is greater than a second thickness T2 adjacent lateral side surface 1214. The taper of body 1200 of wedge 1100 provides, for example, the desired alignment of the medial cuneiform bone or the lateral side of the calcaneus in certain applications. The thinner lateral portion 1220 is sized to be inserted toward an interior portion of a joint or cut bone, and the wider medial portion 1210 is sized to extend toward an exterior of the joint or cut bone. The extent of the taper may depend on the specific deformity or correction desired, such as, for example, a more medially taper for a forefoot or a taper to cause a big toe to point more downward.

Medial portion 1210 includes an outer medial side surface 1212 and an inner medial surface 1214 of body 1200 extending between rounded corners 1230, 1232. Body 1200 of wedge 1100 includes two appendages 1240, 1250 extending from medial portion 1210. Appendages 1240, 1250 with medial portion 1210 form the U-shaped or horseshoe shape of wedge 1100. A central opening 1260 may be located on lateral portion 1220 of wedge body 1200 and formed by inner surfaces 1244, 1254 of appendages 1240, 1252, respectively, and inner surface 1214 of medial portion 1210. Appendage 1240 includes an outer surface 1242 extending from rounded corner 1230 to lateral side 1222. Appendage 1250 includes an outer surface 1252 extending from round corner 1232 to lateral side 1222.

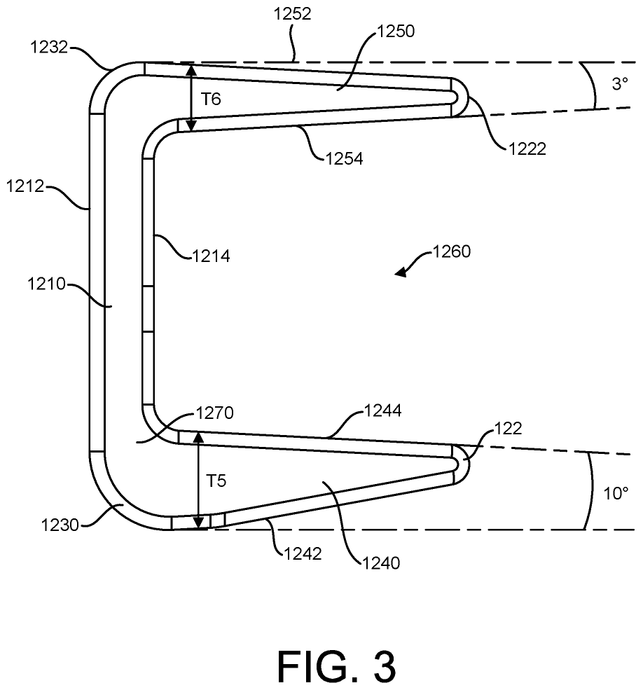

In one example illustrated in FIG. 3, appendage 1240 may be larger taper than appendage 1250. In this example, the shape of bone may make it beneficial for appendage 1240, positioned on, for example, the bottom side of a patient's foot, to have a larger taper than appendage 1250. In one example, the taper of appendage 1240 may be set at ten degrees, while the taper of appendage 1250 is set at three degrees. In order for both appendages 1240, 1250 to come to about the same width and terminate at lateral side 1222 together, appendage 1240 would need to be made thicker than appendage 1250 (e.g. compare T5 and T6 in FIG. 3). In alternative embodiments, both appendages 1240, 1250 may include the same taper, resulting in both having the same thickness, making bone wedge implant 1000 appear symmetrical.

Body 1200 of wedge 1100 also includes a distal side surface 1270 and a proximal side surface 1280 extending between medial side 1212 and lateral side 1222. As illustrated in FIG. 5, distal side surface 1270 extends between medial side 1212 generally in a first plane that extends at a first acute angle A1 relative to a central plane 1110 of wedge 1100. Proximal side surface 1280 also extends between medial side 1212 and lateral side 1222 generally in a second plane that extends at a second acute angle A2 relative to a central plane 1110 of wedge 1100. In one embodiment, first acute angle A1 may be the same as second acute angle A2 to central plane. In alternative embodiment, first acute angle A1 is different than second acute angle A2.

Past wedge designs have been, for example, porous for bone to grow through the wedge body or include teeth or serrated surfaces that contact bone. In these past designs, once bone heals and fuses, the past wedges could not be removed without a saw or disrupting the fused bone. In one example, bone wedge implant 1000 constructed in accordance with one or more aspects of the present disclosure may be designed to have all of the bone contacting surfaces, including distal side surface 1270 and proximal side surface 1280, smooth, neutral and/or drafted away from the face of body 1200 with no undercuts and be non-porous. As a result, even after fusion occurs, bone wedge implant 1000 would be capable of being removed from the bone or joint without having to remove or destroy any bone, similar to, for example, how injection-molded parts are designed to be released out of a mold. In this circumstance, the “mold” is any potential bone growth. Opening 1260 formed in lateral side 1220 of body 1200 also assists in the easier removal of bone wedge implant 1000 from bone or a joint than past wedge designs that form a inner hole surrounded by a continuous, uninterrupted body.

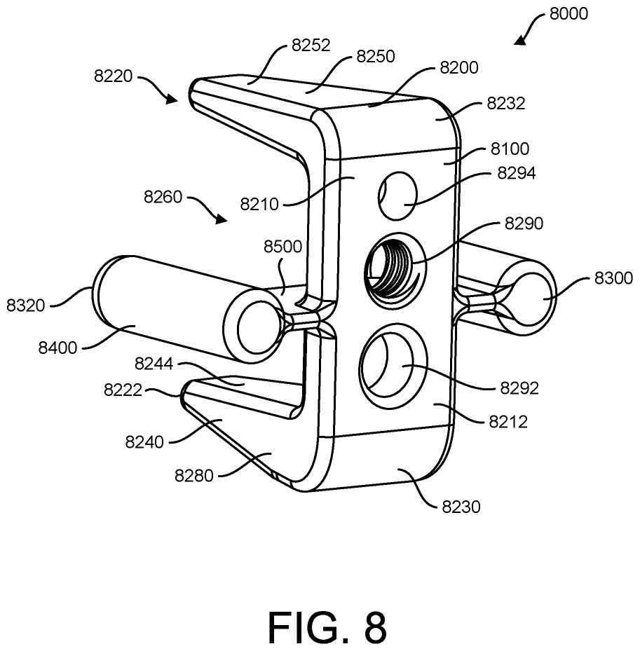

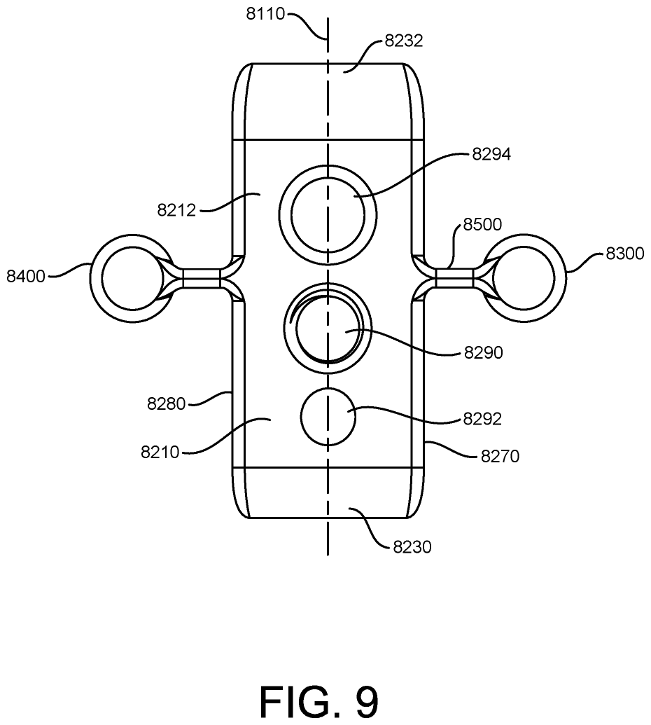

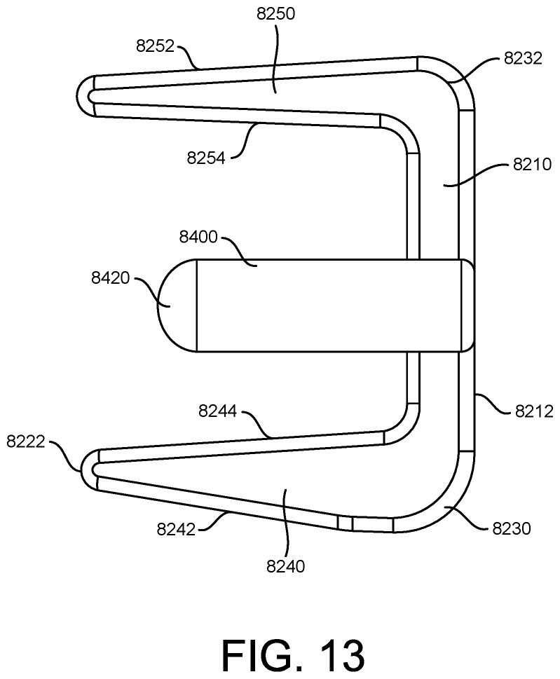

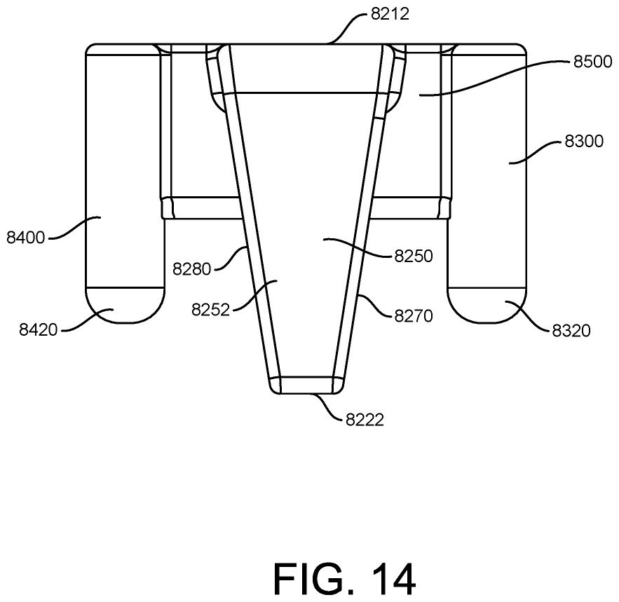

Use of bone wedge implant 1000 illustrated in, for example, FIGS. 1-7, only restricts motion in one plane. However, a person's normal gate while walking resulting in movement of bones in multiple directions. A bone wedge implant 1000 depicted in, for example, FIGS. 1-7 may lack the ability to keep bones from separating away from or shearing across wedge body 1200 in certain conditions. Under certain circumstances, additional stability and support to the surrounding bones are necessary to lock down all planes of motion. In another embodiment illustrated in FIGS. 8-14, bone wedge implant 8000 constructed in accordance with one or more aspects of the present disclosure is provided to provide additional support in multiple planes.

As illustrated in FIGS. 8-14, bone wedge implant 8000 may be generally described as a horseshoe or U-shaped configuration designed to generally conform to an anatomical cross section of, for example, the medial cuneiform, or the lateral side of the calcaneus to provide mechanical support or other bone or joint in which it will be implanted.

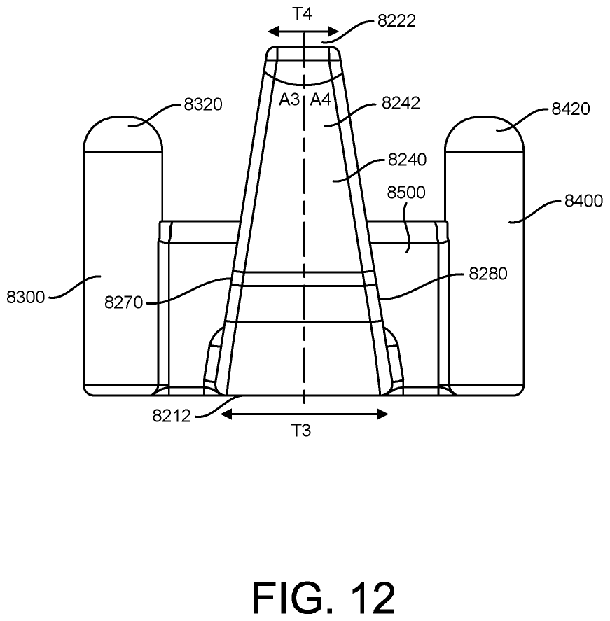

In one example illustrated in FIGS. 8-14, bone wedge implant 8000 includes a wedge 8100 having a body 8200. A top view of bone wedge implant 8000, shown in FIG. 12, shows that body 8200 of wedge 8100 is generally U-shaped or horseshoe shaped with rounded corners 8230, 8232.

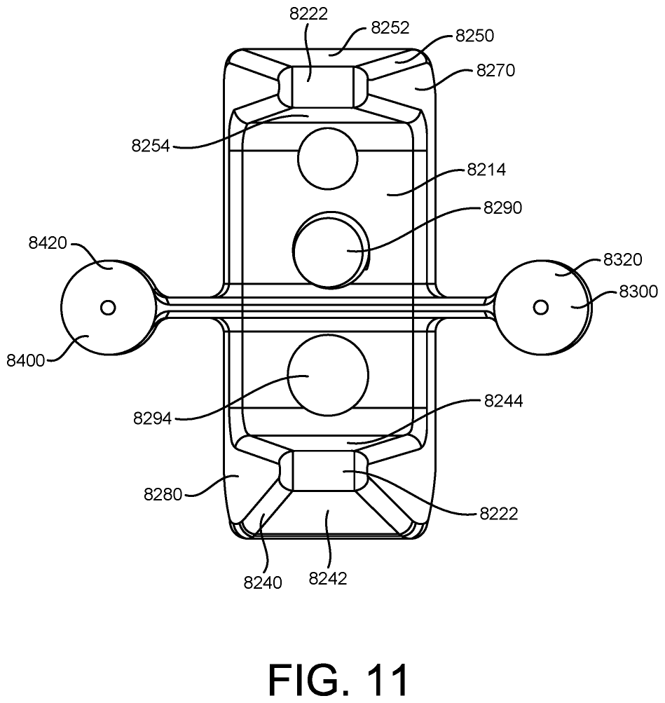

As illustrated in FIGS. 8 and 11, body 8200 of wedge 8100 is tapered from a medial portion 8210 to a lateral portion 8220. In other words, the thickness of body 8200 continuously decreases from medial portion 8210 to lateral portion 8220. For purposes of illustration in FIG. 12, body 8200 of wedge 8100 has a first thickness T3 adjacent medial side surface 8212, which is greater than a second thickness T4 adjacent lateral side surface 8222. The taper of body 8200 of wedge 8100 provides, for example, the desired alignment of the medial cuneiform bone or the lateral side of the calcaneus in certain applications. The thinner lateral portion 8220 is sized to be inserted toward an interior portion of a joint or cut bone, and the wider medial portion 8210 is sized to extend toward an exterior of the joint or cut bone. The extent of the taper may depend on the specific deformity or correction desired, such as, for example, a more medially taper for a forefoot or a taper to cause a big toe to point more downward.

Medial portion 8210 includes an outer medial side surface 8212 of body 8200 extending between rounded corners 8230, 8232. Body 8200 of wedge 8200 includes two appendages 8240, 8250 extending from medial portion 8210 towards lateral side 8222. Appendages 8240, 8250 with medial portion 8210 form the U-shaped or horseshoe shape of wedge 8100. A central opening 8260 may be located on lateral side 8220 of wedge 8100 between inner surfaces 8244, 8254 of appendages 8240, 8250, respectively, and inner surface 8214 of medial portion 8210. Appendage 8240 includes an outer surface 8242 extending from rounded corner 8230 to lateral side 8222. Appendage 8250 includes an outer surface 8254 extending from round corner 8232 to lateral side 8222. Appendage 8250 may be larger than, smaller than or the same size as appendage 8240.

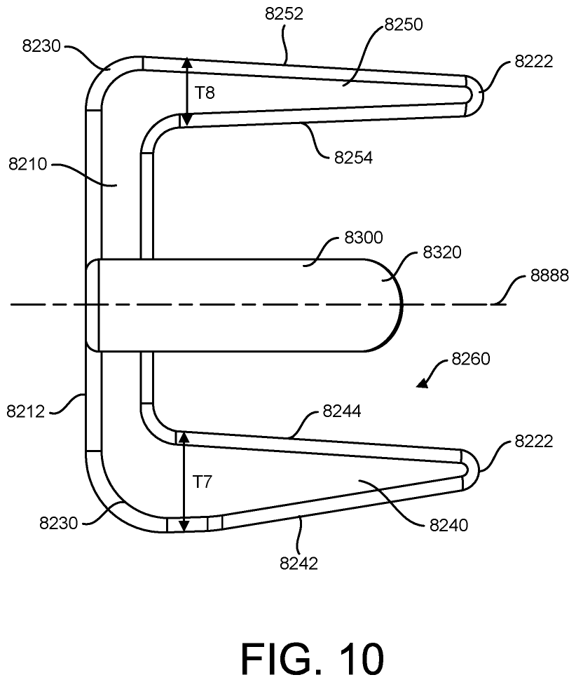

In one example illustrated in FIG. 10, appendage 8240 may be larger taper than appendage 8250. In this example, the shape of bone may make it beneficial for appendage 8240, positioned on, for example, the bottom side of a patient's foot, to have a larger taper than appendage 8250. In one example, the taper of appendage 8240 may be set at ten degrees, while the taper of appendage 8250 is set at three degrees. In order for both appendages 8240, 8250 to come to about the same width and terminate at lateral side 8222 together, appendage 8240 would need to be made thicker than appendage 1250 (e.g. compare T7 and T8 in FIG. 10). In alternative embodiments, both appendages 8240, 8250 may include the same taper, resulting in both having the same thickness, making bone wedge implant 8000 appear symmetrical.

Body 8200 of wedge 8100 also includes a distal side surface 8270 and a proximal side surface 8270 extending between medial side 8212 and lateral side 8222. As illustrated in FIG. 12, distal side surface 8270 extends between medial side 8212 generally in a first plane that extends at a first acute angle A3 relative to a central plane 8110 of wedge 8100. Proximal side surface 8280 also extends between medial side 8212 and lateral side 8222 generally in a second plane that extends at a second acute angle A4 relative to a central plane 8110 of wedge 8100. In one embodiment, first acute angle A3 is the same as second acute angle A4 to central plane 8110. In an alternative embodiment, first acute angle A3 is different than second acute angle A4 to central plane 8110.

In one example, bone wedge implant 8000 may include a first cylindrical post 8300 spaced from distal side surface 8270 and a second cylindrical post 8400 spaced from proximal side surface 8280. First and second cylindrical posts 8300, 8400 may be linked together and to body 8200 of wedge 8100 by a middle bridge 8500. In one embodiment, first and second cylindrical posts 8300, 8400 are solid cylindrical posts and may be inserted and retained within bone without the aid or use of any bone fixation members, e.g. screws. In an alternative embodiment, first cylindrical post 8300 may include a first aperture that may be adapted for receiving a bone-fixation member, such as, for example, a screw, and second cylindrical post 8400 may also include an aperture for receiving a bone-fixation members, such as, for example, another screw. In one example, first and second cylindrical posts 8300, 8400 may be tapered or rounded at their ends 8320, 8420, as illustrated in FIG. 10, to facilitate insertion into bone.

As illustrated in one example shown in FIGS. 8 and 12, middle bridge 8500 may be generally rectangular in shape and orientated perpendicular to central plane 8110 of wedge body 8200. Portions of middle bridge 8500 extend between distal side surface 8270 of body 8200 to first cylindrical post 8300, between proximal side surface 8280 of body 8200 to second cylindrical post 8400, and extend between first cylindrical post 8300 and second cylindrical post 8400. In one example illustrated in FIG. 10, middle bridge 8500 may be offset (e.g. closer to appendage 8240 than appendage 8250 or closer to appendage 8250 than appendage 8240) from the center axis 8888 of opening 8260. This offset configuration may be desirable for a surgeon who may want to insert, for example, a screw at an angle where there is more room. In alternative embodiments, middle bridge 8500 may align with center axis 8888.

First and second cylindrical posts 8300, 8400 of bone wedge implant 8000 assist in, for example, preventing bones bordering wedge 8000 from separating, while middle bridge 8500, for example, eliminates up and down motion and/or bending forces exerted over the osteotomy opening of the bone or joint. A bone wedge implant 8000 constructed accordance with one or more aspects of the present disclosure that includes, for example, first and second cylindrical posts 8300, 8400 and middle bridge 8500 will lock down bone wedge implant 8000 in all planes of motion. In other embodiments, bone wedge implant 8000 may include one or more cylindrical posts on either distal side 8270 and/or proximal side 8280 of wedge body 8200.

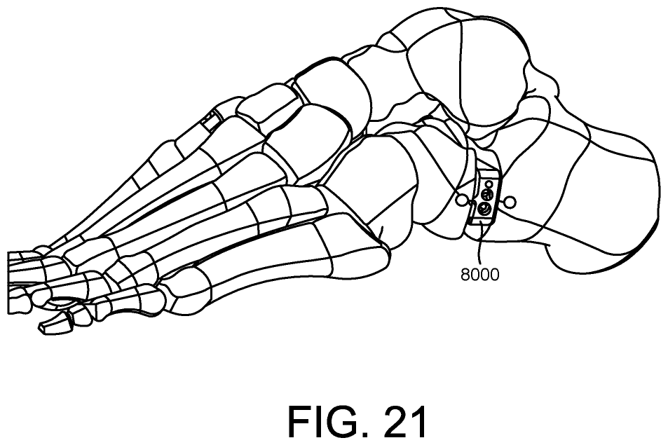

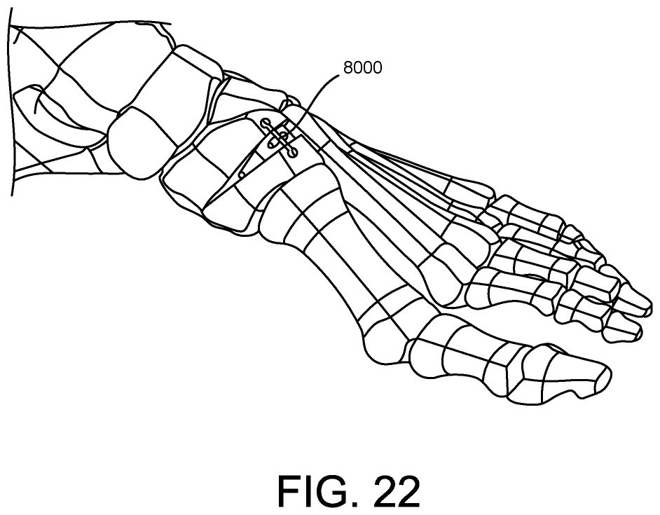

FIG. 21 depicts bone wedge implant 8000 implanted into bone medial cuneiform of a patient and FIG. 22 depicts bone wedge implant 8000 into the lateral side of a calcaneus bone of a patient's foot. As shown in both FIGS. 21 and 22, bone fixation members 2100 are provided through first cylindrical post 8300 and second cylindrical post 8400 when implanted into a joint or boned.

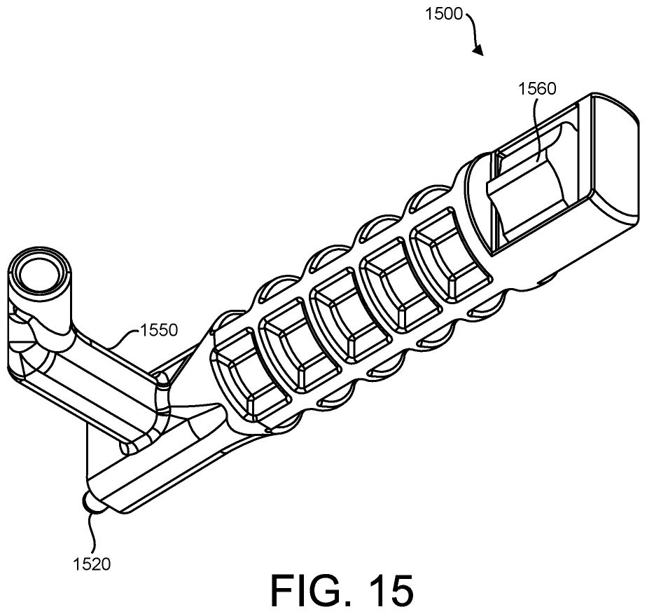

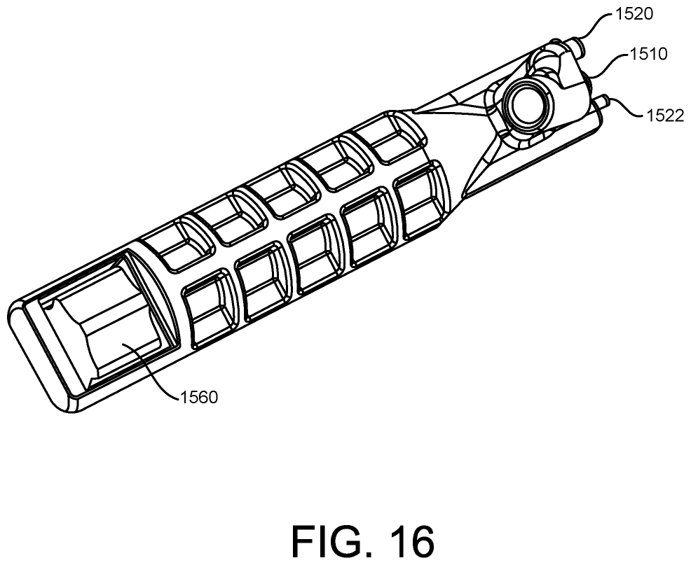

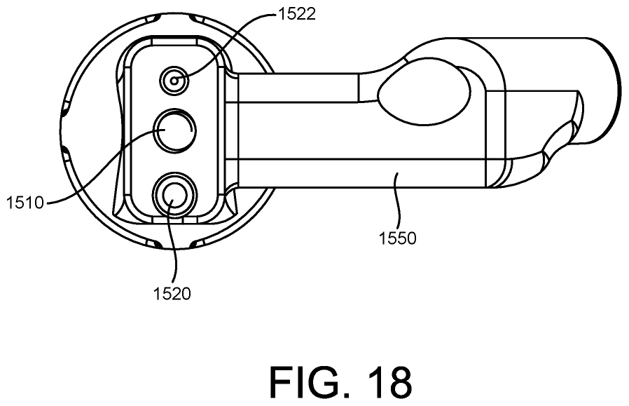

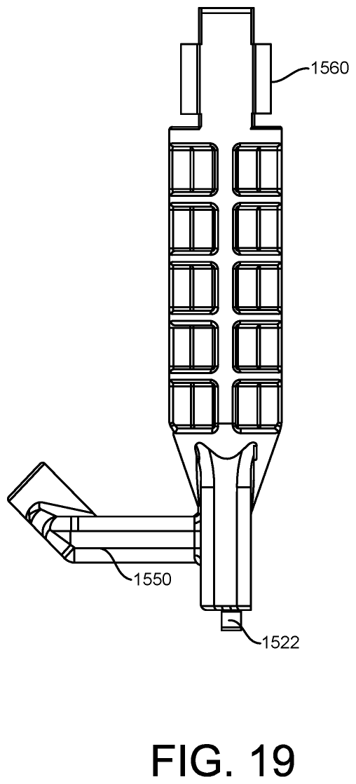

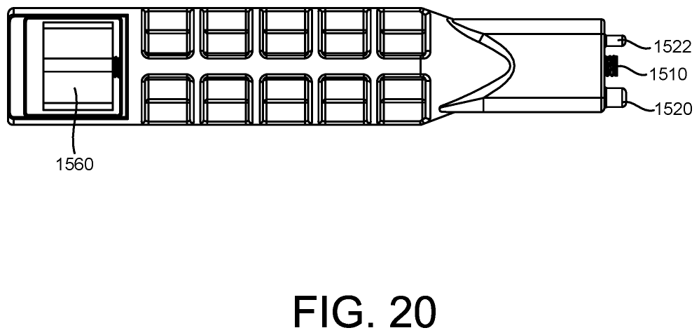

During surgery, an installer instrument may be used by a surgeon to, for example, bone wedge implants into, for example, a cut section of the medial cuneiform bone or into the lateral side of calcaneus bone. FIGS. 15-20 illustrated one example of such an installer instrument 1500. After the wedge 1000 or 8000 is implanted, installer instrument 1500 can be unscrewed and removed from wedge 1000 or 8000. As noted above, the tapered body 1200, 8200 of wedge 1000, 8000 increases the ease of implanting wedge.

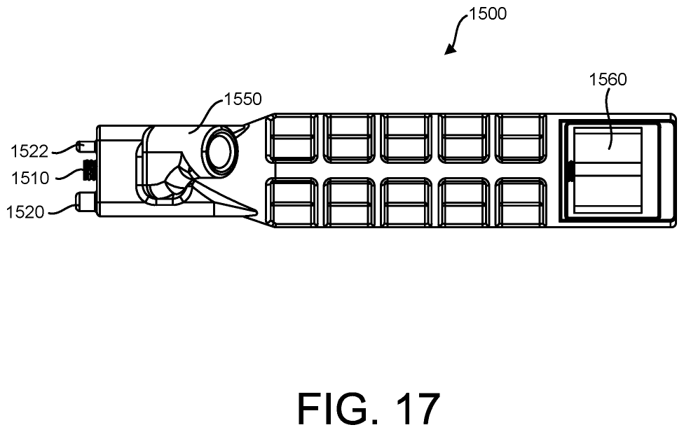

In one example, wedge body 1200 may also include an installation aperture 1290 formed in medial portion 1210 that is sized, shaped or configured for receiving an installer 1500 or other instrument (See FIG. 1). For instance, installation aperture 1290 may include a threaded inner surface with which the installer 1500 may engage during surgical insertion of bone wedge implant. In an alternative embodiment shown in FIGS. 8-14, wedge body 8200 may also include an installation aperture 8290 formed in medial portion 8210 that is sized, shaped or configured for receiving an installer 1500 or other instrument (See FIG. 1). For instance, installation aperture 8290 may include a threaded inner surface with which the installer 1500 may engage during surgical insertion of bone wedge implant. In one example illustrated in FIG. 17, installer instrument 1500 may include a threaded tip 1510 at a distal end thereof for insertion into the threaded openings 1290, 8290 of wedges 1000, 8000, respectively. In one example, threaded tip 1510 of installer instrument 1500 may engage threaded opening 1290, 8290 by slidably engaging threaded tip 1510 to threaded opening 1290, 8290 and rotating or turning a knob 1560 at the proximal end of installer instrument 1500 to engage the threads of threaded tip 1510 to the threads of threaded opening 1290, 8290.

As shown in FIG. 17, instrument 1500 may also include one or more pins 1520, 1522 extending from distal end configured to couple to first and second pin openings 1292, 1294 formed in medial portion 1210 of body 1200 of wedge 1000 or to first and second pin openings 8292, 8294 formed in medial portion 8210 of body 8200 of wedge 8000. For example, pins 1250, 1252 are configured to couple to first and second pin openings 1292, 1294 of wedge 1000, or 8292, 8294 of wedge 8000, may be used to keep instrument and bone wedge implant in a correct orientation while preventing spinning that may occur with the use of only one installation aperture 1290, 8290. Installer instrument 1500 may also include an arm 1550 extending from a main body that could, for example, guide a transverse screw member across the wedge body without hitting first and second cylindrical posts or the sides of wedge body.

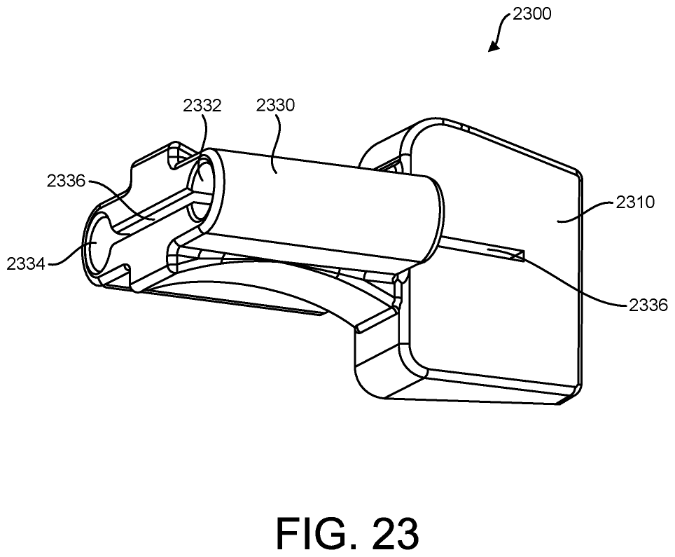

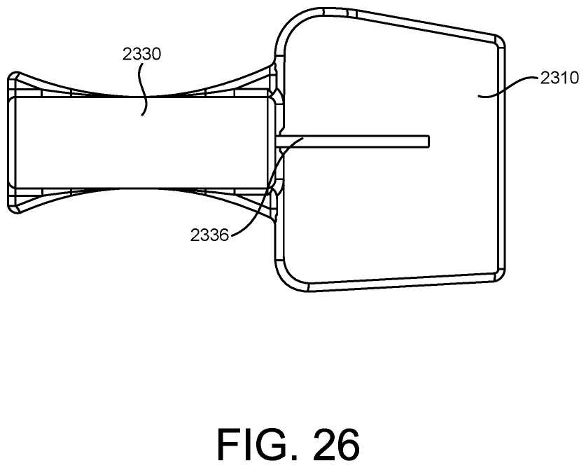

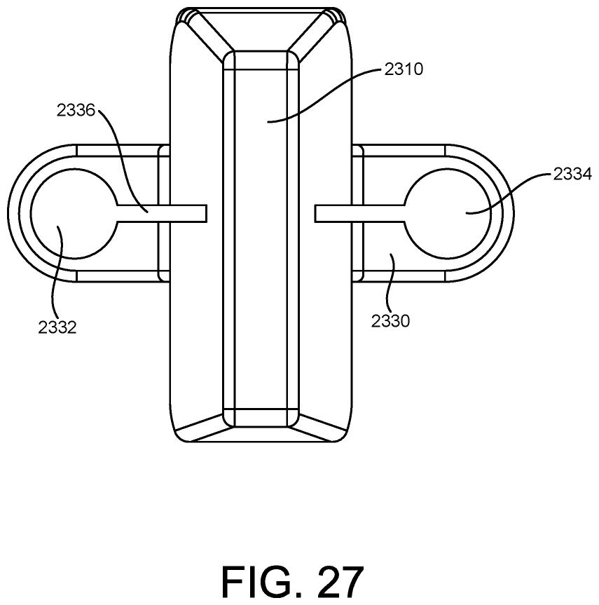

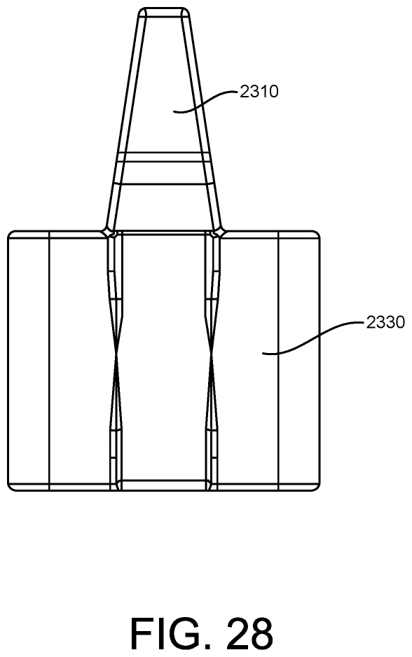

FIGS. 23-29 illustrate one example of a trial and preparation instrument 2300 for facilitating bone preparation of the bone or joint in which, for example, bone wedge implant 8000 illustrated in FIGS. 8-14 may be implanted. In one example illustrated in FIGS. 23-28, trial and preparation instrument 2300 includes a trial portion 2310 on one side and a guide body 2330 on the other side. In one example, trial portion 2310 mimics or corresponds to the tapered profile of body 8200 of wedge 8100 to be implanted. In alternative embodiments, trial portion 2310 may be removeable affixed to guide body 2330 to, for example, accommodate multiple sizes for each desired taper of wedge body 8200 provided. Trial portion 2310 may also act as a trial so the surgeon or clinician can ensure that the proper bone wedge implant is selected.

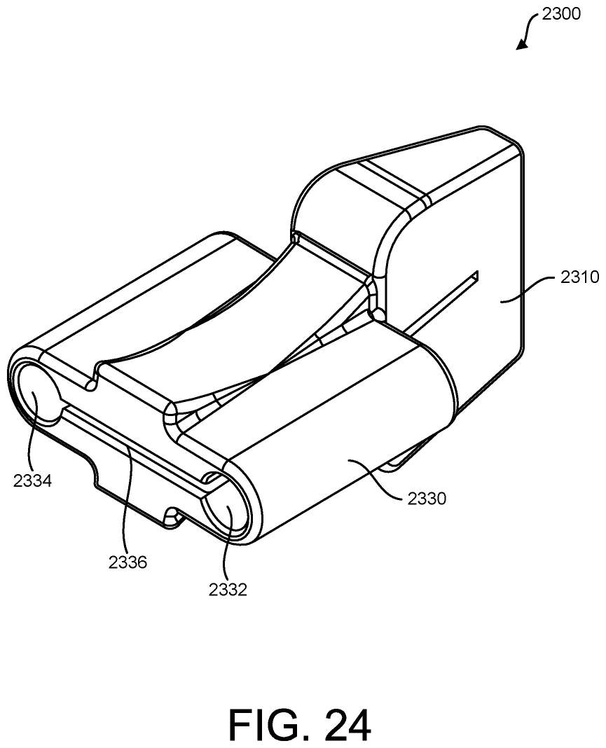

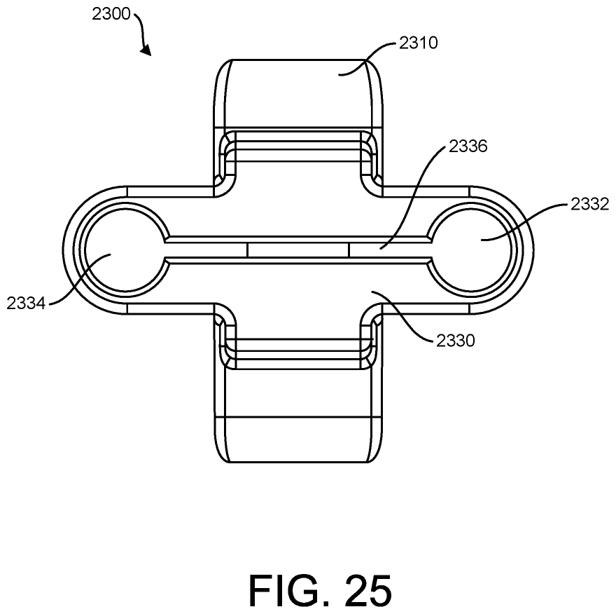

In one example, guide body 2330 of trial and preparation instrument 2300 acts as a guide for making the drill and saw cuts for first and second cylindrical posts 8300, 8400 and middle bridge 8500 on bone wedge implant 8000. As illustrated in FIGS. 24-25, guide body 2330 comprises first and second bores 2332, 2334 for receiving, for example, a drill bit as well as a saw or milling slot 2336 extending between the first and second bores 2332, 2334. First and second bores 2332, 2334 are sized and configured to drill holes in the bone to mate or correspond with first and second cylindrical posts 8300, 8400, respectively, of bone wedge implant 8000, while milling slot 2336 is sized and configured to saw or mill a channel in bone to mate or correspond with middle bridge 8500 of bone wedge implant 8000.

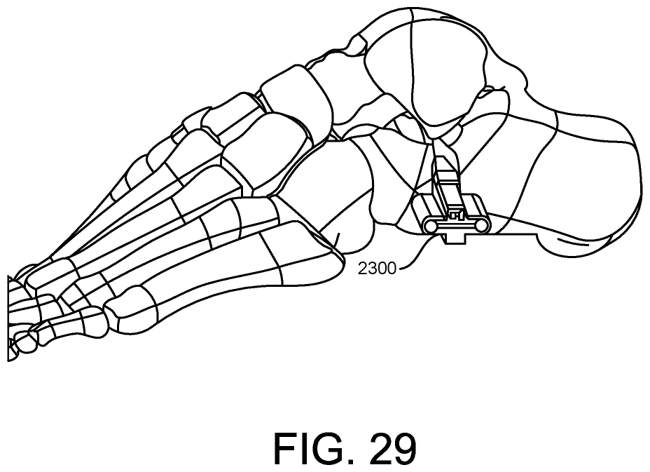

During surgery, trial and preparation instrument 2300 is positioned such as to bridge or straddle the osteotomy opening, wherein first bore 2332 is arranged on the distal side of the osteotomy opening and second bore 2334 is arranged on the proximal side of the osteotomy, as illustrated, for example, in FIG. 29. During surgery, a surgeon or clinician would insert trial portion 2310 into the osteotomy until satisfied with the positioning. Then, a surgeon or clinician would use a drill to create a first bone bore using first bore 2332 of guide body 2330 and a second bone bore using second bore 2334 of guide body 2330 to, for example a defined depth. A surgeon or clinician would also use a saw or milling tool to saw or mill bone through saw or milling slot 2336 to, for example, form a channel linking link first bone bore and second bone bore.

After first bone bore, second bone bore and channel are cut into the bone(s), trial and preparation instrument 2300 is removed and the appropriate bone wedge implant 8000 is implanted. The appropriate bone wedge implant 8000 implanted would include a wedge body taper that mimics the trial portion 2310 of the trial and preparation instrument 2300 used. Bone wedge implant 8000 is implanted into the bone(s) by, for example, aligning first cylindrical post 8300 with first bone bore formed by first bore 2332 of guide body 2330, aligning second cylindrical post 8400 with second bone bore formed by second bore 2334 of guide body 2330 and middle bridge 8500 with channel formed by milling slot 2335 of guide body 2330. The bone wedge implant 8000 would then be inserted in the bone bed using, for example, an installer instrument 1500 such as that depicted in FIGS. 15-20. Once installed, bone wedge implant 8000 bridges the osteotomy opening, and transfers the forces caused by bodyweight of and on the surrounding bone(s) over the load-sharing construct of wedge, that includes the wedge body 8200, first and second cylindrical posts 8300, 8400 and middle bridge 8500.

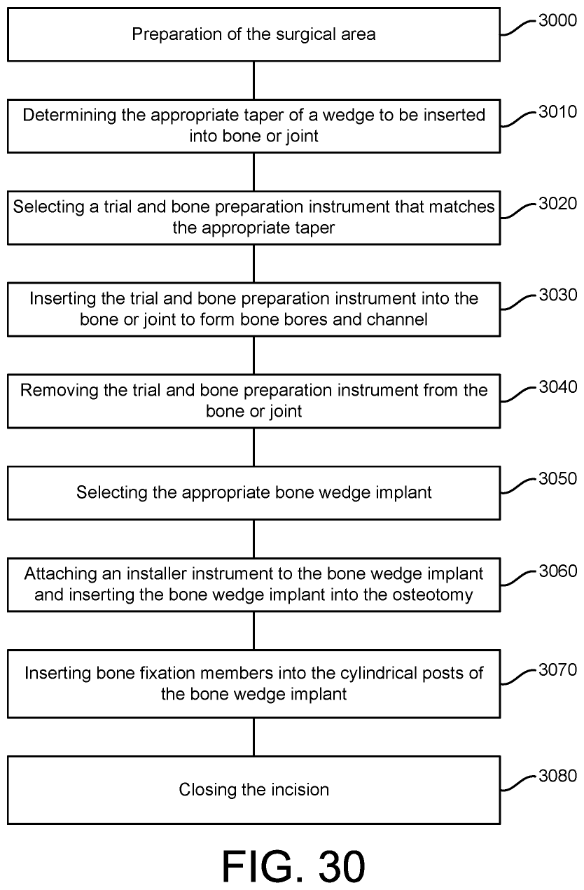

FIG. 30 depicts one example of a surgical method of implanting a bone wedge implant 8000 using a trial and bone preparation instrument 2500 and an installer instrument 1500, constructed in accordance with one or more aspect of the present disclosure. The surgical method may include, for example, a step 3000 of preparing a surgical area. Step 3000 of preparing the surgical area may include, for example, surgically exposing a portion of the patient's foot to expose the patient's medial cuneiform and/or the patient's calcaneus, making an incision and preparing the bone or joint for bone wedge implant 8000. As part of, for example, step 3000, a surgeon or clinician may distract the joint or bone space. The surgical method may include a step 3010 to determine the appropriate taper of bone wedge implant 8000 to be inserted into the distracted bone or joint. Step 3020 may include selecting an appropriate trial portion 2310 of trial and preparation instrument 2300 that will mimic the taper of wedge body 8200. Step 3030 may include using a trial and preparation instrument 2300, such as the instrument depicted in FIGS. 23-28, to prepare the bone bed for insertion of a bone wedge implant 8300 constructed in accordance with one or more aspects of the present disclosure. Step 3030 may also include forming the bone bores and channel using bore apertures 2332, 2334 and milling slot 2336 of guide body 2330. Step 3040 may include removing the trial and bone preparation instrument from the bone or joint. Step 3050 may include selecting the appropriate bone wedge implant. This step may include making sure the taper of body 8200 matches the taper portion 2310. Step 3060 may include attaching an installer instrument 1500, such as the one depicted in FIGS. 15-20 to body 8200 to facilitate insertion of bone wedge implant 800 into the prepared bone bed of the osteotomy created by trial and preparation instrument 2300. In this step, pins 1520, 1522 may be inserted into pin holes 8292, 8294 in body 8200 and threaded tip 1510 of installer 1500 may be advanced and threaded into threaded aperture 8290 by turning knob 1560. Step 3060 may also include inserting bone wedge implant 8000 into the osteotomy. In one embodiment, bone wedge implant 8000 may be further secured in place, in step 3070, by inserting bone fixation members through first and second apertures of extending through first and second cylindrical posts 8300, 8400, respectively. Finally, step 3080 may include closing the patient's incision.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. An osteotomy implant device, said osteotomy implant device comprising:
a tapered wedge, said tapered wedge including a medial portion and a lateral portion, the lateral portion including an outer lateral side, wherein the lateral portion includes a first appendage and a second appendage, the first appendage extending from a first end of the medial portion to the lateral side, the second appendage extending from a second end of medial portion to the lateral side, wherein the first appendage, the medial portion and the second appendage form a U-shaped body, the U-shaped body defining an outer perimeter side, an inner perimeter side, a distal side extending between the outer perimeter side and the inner perimeter side, and a proximal side opposite the distal side and extending between the outer perimeter side and the inner perimeter side, the inner perimeter side defining a central opening, the distal side extending along the first and second appendages and the medial portion and the proximal outer side extending along the first and second appendages and the medial portion;
a first cylindrical post, said first cylindrical post spaced from the distal side of the U-shaped body of said tapered wedge;
a second cylindrical post, said second cylindrical post spaced from the proximal side of the U-shaped body of said tapered wedge; and
a middle bridge, said middle bridge spanning lengthwise from said first cylindrical post to said second cylindrical post, a first portion of said middle bridge extending from the distal side of said tapered wedge to the first cylindrical post, and a second portion of said middle bridge extending from the proximal side of said tapered wedge to the second cylindrical post.

2. The osteotomy implant device of claim 1, wherein the osteotomy implant device is configured to be inserted into an osteotomy using an installer instrument removeably affixed to the medial portion of said tapered wedge.

3. The osteotomy implant device of claim 1, wherein the medial portion of the tapered wedge is thicker than the lateral portion of the tapered wedge.

4. The osteotomy implant device of claim 1, wherein the first cylindrical post includes a rounded end to facilitate insertion into bone.

5. The osteotomy implant system, said osteotomy implant system comprising:
an osteotomy implant device, said osteotomy implant device including:
a tapered wedge, said tapered wedge including a medial portion and a lateral portion, the lateral portion including an outer lateral side, wherein the lateral portion includes a first appendage and a second appendage, the first appendage extending from a first end of the medial portion to the lateral side, the second appendage extending form a second end of medial portion to the lateral side, wherein the first appendage, the medial portion and the second appendage form a U-shaped body, the U-shaped body defining an outer perimeter side, an inner perimeter side, a distal side extending between the outer perimeter side and the inner perimeter side, and a proximal side opposite the distal side and extending between the outer perimeter side and the inner perimeter side, the inner perimeter side defining a central opening, the distal side extending along the first and second appendages and the medial portion and the proximal outer side extending along the first and second appendages and the medial portion;
a first cylindrical post, said first cylindrical post spaced from the distal side of the U-shaped body of said tapered wedge;
a second cylindrical post, said second cylindrical post spaced from the proximal side of the U-shaped body of said tapered wedge;
a middle bridge, said middle bridge spanning lengthwise from said first cylindrical post to said second cylindrical post, a first portion of said middle bridge extending from the distal side of said tapered wedge to the first cylindrical post, and a second portion of said middle bridge extending from the proximal side of said tapered wedge to the second cylindrical post;
an installer instrument, said installer instrument removeably coupled to the medial portion of said tapered wedge to facilitate insertion of said tapered wedge into an osteotomy; and
a trial and bone preparation instrument, said trial and bone preparation instrument including a guide body and a trial portion, the trial portion including a tapered wedge portion that mimics said tapered wedge for insertion into the osteotomy, the guide body including a first bore, a second bore and a milling slot extending between the first bore and the second bore, the first bore, the second bore and the milling slot configured to prepare the osteotomy for said osteotomy implant device.

6. An osteotomy implant device, said osteotomy implant device comprising:
a tapered wedge, said tapered wedge including:
a medial portion, the medial portion including a first end and a second end;
a first appendage, the first appendage extending from the first end of the medial portion;
a second appendage, the second appendage extending from the second end of the medial portion,
wherein the medial portion, the first appendage and the second appendage form a U-shaped body and define a central opening, the U-shaped body including a first surface defining a first edge and a second edge, a second surface defining a third edge and a fourth edge, a distal side surface extending between the first edge of the first surface and the first edge of the second surface along the first appendage, the medial portion and the second appendage, and a proximal side extending between the second edge of the first surface and the second edge of the second surface along the first appendage, the medial portion and the second appendage;
a first cylindrical post, said first cylindrical post spaced from a portion of the distal side of the U-shaped body of said tapered wedge;
a second cylindrical post, said second cylindrical post spaced from the proximal side of the U-shaped body of said tapered wedge; and
a middle bridge, said middle bridge spanning lengthwise from said first cylindrical post to said second cylindrical post, a first portion of said middle bridge extending from the distal side of the medial side of the U-shaped body of said tapered wedge to the first cylindrical post, and a second portion of said middle bridge extending from the proximal side of the medial portion of the U-shaped body of said tapered wedge to the second cylindrical post.

7. A method for wedge osteotomy of a foot, said method comprising the steps of:
exposing an osteotomy site of the foot;
providing an osteotomy implant device, said osteotomy implant device comprising:
a tapered wedge, said tapered wedge including a medial portion and a lateral portion, the lateral portion including an outer lateral side, wherein the lateral portion includes a first appendage and a second appendage, the first appendage extending from a first end of the medial portion to the lateral side, the second appendage extending form a second end of medial portion to the lateral side, wherein the first appendage, the medial portion and the second appendage form a U-shaped body, the U-shaped body defining an outer perimeter side, an inner perimeter side, a distal side extending between the outer perimeter side and the inner perimeter side, and a proximal side opposite the distal side and extending between the outer perimeter side and the inner perimeter side, the inner perimeter side defining a central opening, the distal side extending along the first and second appendages and the medial portion and the proximal outer side extending along the first and second appendages and the medial portion;
a first cylindrical post, said first cylindrical post spaced from the distal side of the U-shaped body of said tapered wedge;
a second cylindrical post, said second cylindrical post spaced from the proximal side of the U-shaped body of said tapered wedge; and
a middle bridge, said middle bridge spanning lengthwise from said first cylindrical post to said second cylindrical post, a first portion of said middle bridge extending from the distal side of said tapered wedge to the first cylindrical post, and a second portion of said middle bridge extending from the proximal side of said tapered wedge to the second cylindrical post;
inserting the osteotomy implant device across the osteotomy site.
